(19) United States
(12) Patent Application Publication
(10) Pub. No.: US 2015/0343190 A1
(43) Pub. Date: Dec. 3, 2015

(54) METHOD OF OXYGEN SATURATION FOR WOUND TREATMENT

(71) Applicant: Prosper Brands LLC, Brooklyn, NY (US)

(72) Inventors: Craig Glassford, New Maryland (CA); James Snider, Island View (CA); John Archibald, Grimsby (CA)

(21) Appl. No.: 14/639,000

(22) Filed: Mar. 4, 2015

Related U.S. Application Data
(60) Provisional application No. 61/966,851, filed on Mar. 5, 2014.

(51) Int. Cl. A61M 37/00

(52) U.S. Cl. CPC A61M 37/00

(57) ABSTRACT

A method of administering oxygen to a body site comprising: providing an aqueous solution having a dissolved oxygen level of 18 mg/L or greater; containing the aqueous solution in a receptacle suitable for at least partially submerging a portion of a body part; applying the aqueous solution to the body part for a period of time allowing for oxygen transfer between the aqueous solution and at least some of the tissue over the body part.

### CROSS-REFERENCE TO RELATED APPLICATION(S)

This application claims the benefit of priority under 35 U.S.C. §120 to U.S. Prov. App. No. 61/966,851, entitled Method and Apparatus for Wound Treatment, filed Mar. 5, 2014, the contents of which are incorporated herein by reference in their entirety.

### TECHNICAL FIELD

This invention relates to a method and apparatus for delivering a gas to tissue beneath the skin. In particular, the invention relates to a method and apparatus for microvascular wound treatment by the bulk transfer of dissolved oxygen through human skin into subcutaneous tissue of the foot and lower leg.

### BACKGROUND

Approximately 1 in 17 persons are diagnosed with diabetes. Diabetes is considered a metabolic disease yet vascular problems are what cause more diabetic complications that reduce quality of life and longevity. One serious diabetic complication is the diabetic foot ulcer. Diabetic foot ulcers are any break in the skin although they usually extend through the full thickness of the skin and can involve deeper structures of the foot such as tendon and bone. The ulcers are painful, recurrent and slow to heal. Between 15 and 25% of all diabetics will be affected by foot ulcers in their lifetime and the clinical endpoint is often amputation of the affected toes, feet and lower limbs.

Hyperbaric oxygen therapy (HBOT) has been used with some success to treat diabetic foot ulcers. Patients breathe 100% oxygen while sitting in a special chamber that can be pressurized. The procedure increases the amount of oxygen in their blood so that more oxygen can be delivered to the tissues around the foot ulcer. Oxygen supports the energetic requirement of the wound repair process. In some studies, patients treated with HBOT were found to have faster ulcer healing rates and they had fewer ulcers that led to amputation. Unfortunately, HBOT is not readily available treatment and breathing oxygen in high pressure environments poses some safety risk to the patient.

### SUMMARY

Embodiments disclosed herein address the above stated needs by providing a method and apparatus to facilitate oxygen absorption in the tissue of an extremity of a mammal by immersing the extremity in an oxygen infused liquid.

In one example aspect of the invention a method of administering oxygen to a body site comprises: providing an aqueous solution having a dissolved oxygen level of 18 mg/L or greater; containing the aqueous solution in a receptacle suitable for at least partially submerging a portion of a body part; applying the aqueous solution to the body part for a period of time allowing for oxygen transfer between the aqueous solution and at least some of the tissue over the body part; removing the aqueous solution from contact with the body part, wherein the transcutaneous partial pressure of oxygen within a portion of the body part is elevated.

In another example aspect of the invention an apparatus for wound treatment comprises: a vessel shaped to accommodate a limb or extremity of a mammal, the vessel having sufficient volume to hold at least 0.5 liters of an aqueous solution in addition to the limb or extremity; and an aqueous solution having a dissolved oxygen level of 18 mg/L or greater.

Other aspects of the invention may include one or more of the following features. The aqueous solution has a dissolved oxygen content of at least 35 mg/L. The oxygen is absorbed into the body part at a rate of 0.7 μmole·cm²·min⁻¹ or greater. The aqueous solution has an oxygen content of at least 45 mg/L and contact is maintained between the aqueous solution and the body part for at least 20 minutes. The aqueous solution is an oxygen infused NaCl solution. The vessel further comprises an intake and an outtake for circulating the aqueous solution about the vessel. The vessel is a water tight boot sized to hold 1.0 or more liters of an aqueous solution having a dissolved oxygen level of 30 mg/L or more. The vessel may be closed in a manner to prevent leakage or spillage of the aqueous solution.

Aspects, embodiments and implementations provide the advantage of being able to transport Oxygen to a wound site independently of the vascular system, thereby benefiting the healing process.

### BRIEF DESCRIPTION OF THE DRAWINGS

FIGS. 1A-C testing procedures consistent with an example embodiment of the present invention;

FIGS. 2A-C illustrate PO2 at various times of an example method of the present invention;

FIGS. 3A-H illustrate treatment results comparing tap water and oxygenated water, in accordance with example embodiments of the present invention;

FIGS. 4A-C illustrate treatment results comparing tap water and oxygenated water, in accordance with example embodiments of the present invention;

FIG. 5 illustrates PO2 at various times and locations of treatment in accordance with example embodiments of the present invention;

FIGS. 6A-C illustrate PO2 at various times and locations of treatment in accordance with example embodiments of the present invention;

FIGS. 7A-C illustrate PO2 at various times and locations of treatment in accordance with example embodiments of the present invention;

FIG. 8 illustrates changes in blood perfusion resulting from oxygen-infused water;

FIG. 9 illustrates changes in blood perfusion resulting from oxygen-infused water;

FIG. 10 illustrates PO2 at various times and locations of treatment in accordance with example embodiments of the present invention; and

FIG. 11 illustrates PO2 at various times and locations of treatment in accordance with example embodiments of the present invention.

### DETAILED DESCRIPTION

Oxygen saturated water used in conjunction with a treatment regime for certain wounds, including diabetic skin lesions and ulcers has been found to be effective. Oxygenation of water or other fluids may be performed using an oxygenation device manufactured by inVentures Technologies Incorporated, which is described in U.S. Pat. No. 7,537,200 (the contents of which are incorporated herein in their entirety). The device significantly increased the amount of oxygen that can be dissolved into aqueous solution at normal barometric pressure. The device in effect creates a hyperbaric oxygen environment in the water. Since the oxygen partial pressure in the water is approximately 25 times greater than the oxygen partial pressure of human tissue, it is possible for human tissue to absorb oxygen from the water when submerged. Skin is a semi-permeable membrane and diffusion of gas across the skin surface is in part dependent on a pressure difference for the gas on either side of the skin. When sufficient oxygen is absorbed into the tissues of the foot from oxygen-infused water, devices and therapies for use in treatment of disease conditions are possible. Example disease conditions include diabetic foot ulcers that occur when the circulatory system is unable to provide adequate oxygen to the epidermal layers.

Experiments were conducted to determine whether it is possible to detect the transfer of oxygen from oxygen-infused water into tissues of the foot.

### Experimental

A series of studies were conducted using young and old adult subjects in order to determine whether there are any age-related differences in the amount of oxygen that can be absorbed from the water.

### General Design

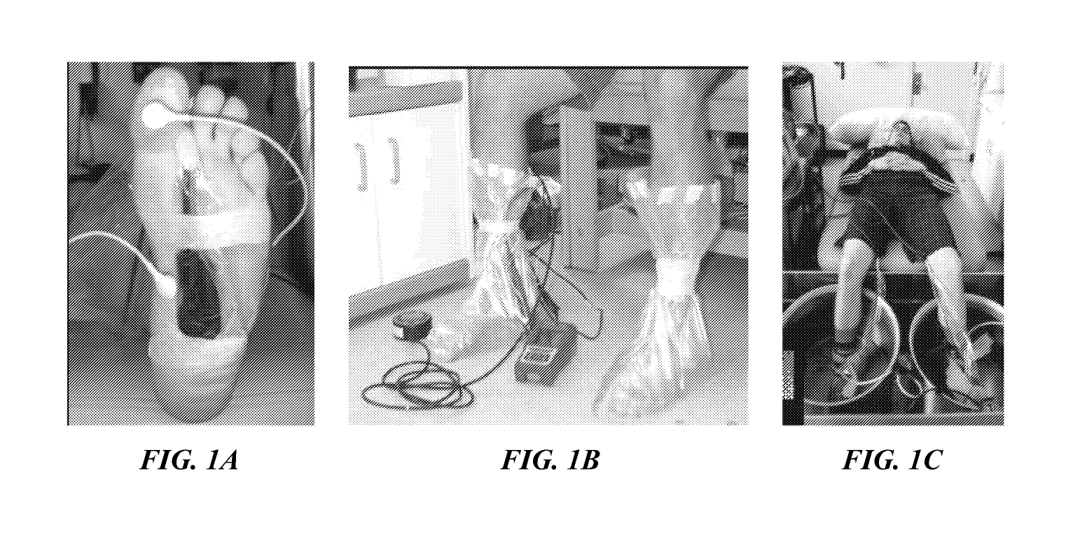

To study the effect of oxygen-infused water on the foot, all subjects were asked to rest quietly in the supine position on a padded bench with the room temperature maintained between 22 and 25° C. Near infrared spectroscopy NIRS (black) and transcutaneous partial pressure of oxygen tcPO₂ (white) sensors were placed on both of the subject's feet and the tcPO₂ was recorded for 15 minutes to allow for stabilization of the measurement (FIG. 1A). The tcPO₂ electrodes were removed and the subject slid forward on the table so that the lower limbs could freely hang from the table edge and suspend each foot in a 50L container filled with tepid tap water (FIG. 1C). The experimental treatment with oxygen-infused water was initiated by rapidly exchanging the tap water with temperature matched oxygen-infused water in one container. Treatment with oxygen-infused water typically lasted 30 minutes. At the end of treatment, the subject's feet were removed from the bath, dried and refitted with tcPO₂ electrodes. Post-treatment tcPO₂ measurements were recorded for 20 minutes. For comparison, tcPO₂ was measured in some subjects before and after breathing 100% O₂ for 30 minutes in place of the water treatment. In some experiments, ischemic hypoxia was used to study the treatment effect. In these experiments, blood flow to feet resting in tap water was arrested for 10 to 12 minutes. The tap water in one container was then rapidly exchanged with oxygen-infused water for 5 minutes before restoring blood flow to both feet. FIG. 1B shows experiments where the foot was soaked in 1 L of water in order to determine O₂ absorption rates and to test the utility of using plastic boot covers 5 in the home-based treatment protocol.

### Direct Measurement of PO₂ in Skin

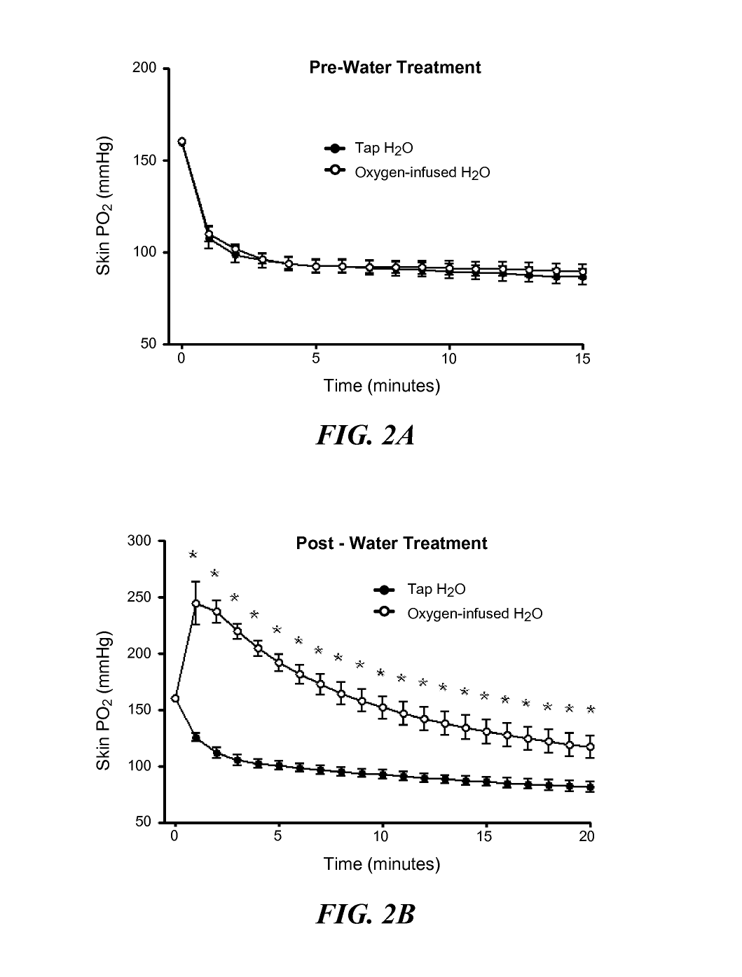

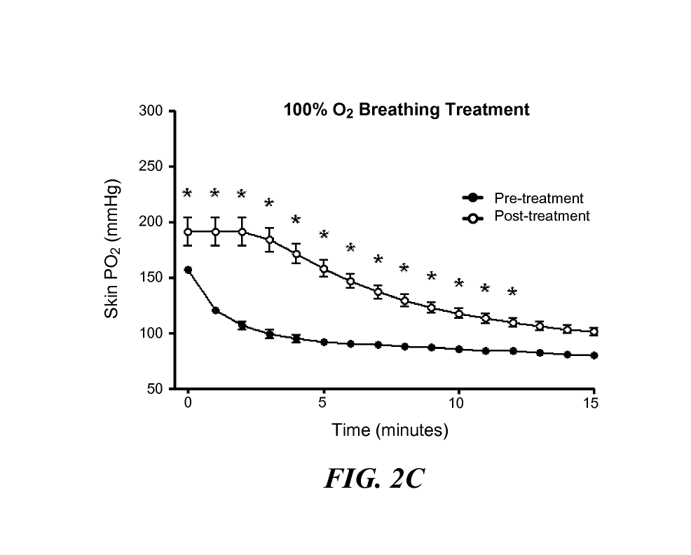

FIGS. 2A-C show the PO₂ measured in the plantar surface of the big toe of 8 young (21 to 40 yrs) adults before treatment with tap water or oxygen-infused water (FIG. 2A). Prior to treatment, tcPO₂ declined from 159.7±0.3 to 86.7±4.3 mmHg and from 160.8±0.8 to 89.5±3.9 mmHg over 15 minutes for the tap water and oxygen-infused water treatment groups respectively (FIG. 2A). After 30 minutes of soaking in tap water (dissolved O₂ (DO) 1.7±0.6 mg·L⁻¹; temp 31.9±0.8° C.), the tcPO₂ probe was reconnected and the tcPO₂ recorded declined steadily from an initial value of 160.3±0.4 to 81.7±4.9 mmHg at 20 minutes post treatment. Significantly higher tcPO2 values were measured in feet treated with oxygen-infused water (DO 63.3±01.4 mg·L⁻¹; temp 33.8±1.4° C.). The tcPO₂ increased from 160.2±0.4 to 244.8±18.8 within 1 minute. By 20 minutes post-treatment the tcPO₂ was 117.3±10.1 mmHg in oxygen-infused water treated feet (FIG. 2B). For comparison, the tcPO₂ on the plantar surface of 20 the big toe was measured before (pre) and after (post) 30 minutes of inspiring 100% oxygen in 5 subjects. The pre-treatment tcPO₂ started at 157.7±1.8 mmHg and declined to 80.4±2.3 mmHg over 15 minutes. After 100% O₂ breathing, tcPO₂ was significantly higher at 191.5±12.8 mmHg. The tcPO₂ 4 values declined to 99.8±3.6 mmHg over the 15 minute interval after 100% O₂ was stopped (FIG. 2C). Higher skin tcPO₂ values were recorded in skin treated with oxygen-infused water than were recorded in subjects breathing 100% O₂ for the same length of time.

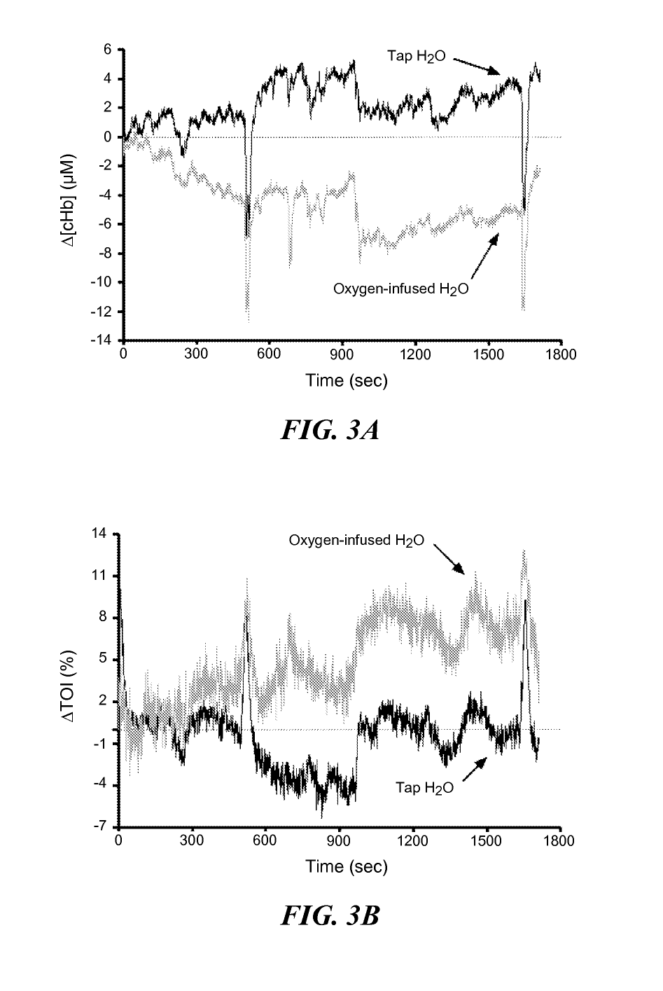

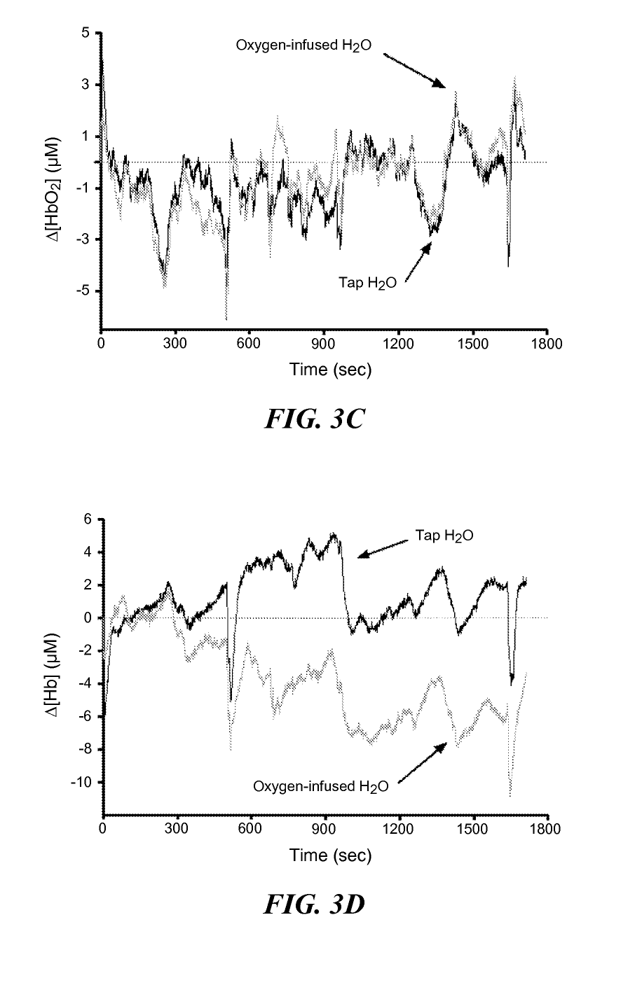

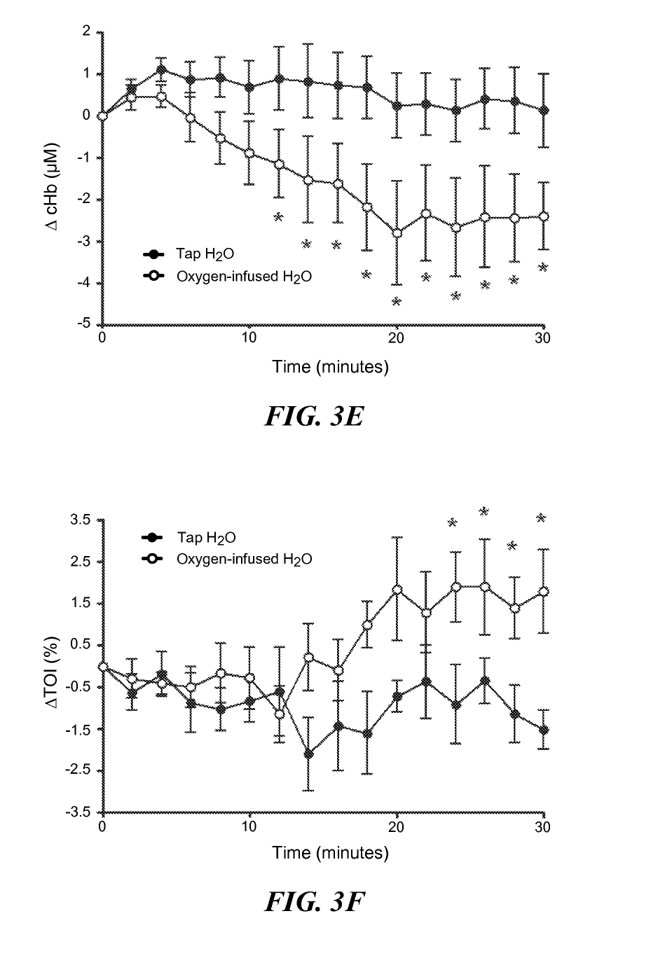

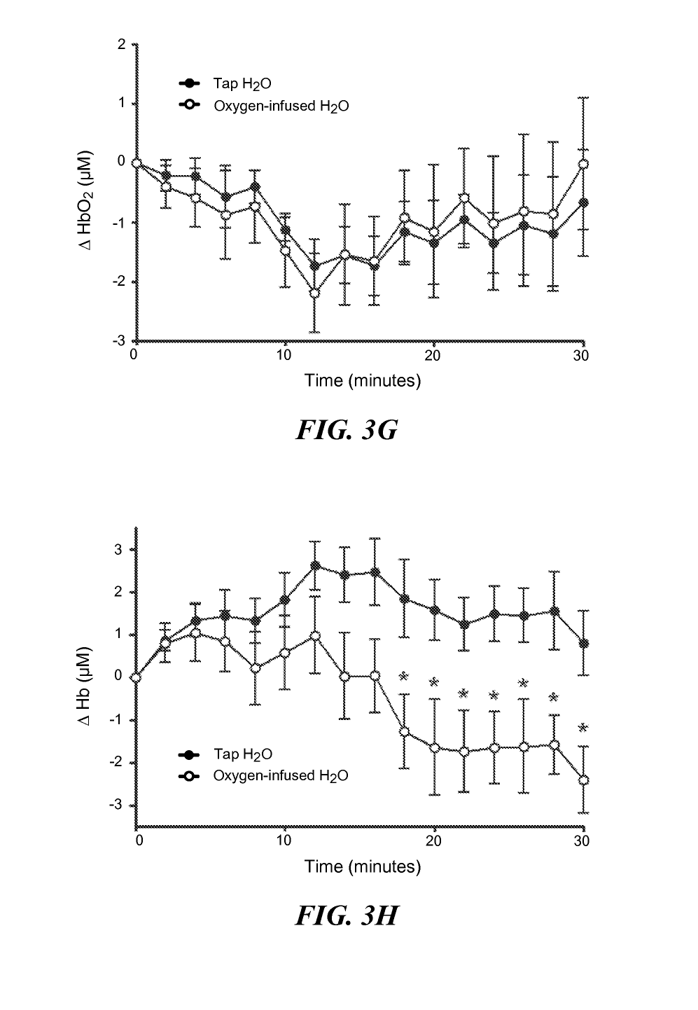

These data support by NIRS recordings made during treatment with either tap water or oxygen-infused water. FIGS. 3A-H show original data from one subject recorded during treatment (left panels) and summary data for 13 individuals (right panels).

NIRS measurements were recorded during treatment with tap water and 10 oxygen-infused water. FIGS. 3A and 3E show that the cHb (cHb: total haemoglobin; represents blood volume) decreased significantly more during treatment with oxygen-infused water (SO 42.9±1.4 mg·L⁻¹; temp 30.8±1.1° C.) than with tap water (DO 2.1±0.3; temp 30.2±0.9° C.). The reduced cHb resulted from a decrease in deoxygenated Hb rather than from a change in 15 oxygenated Hb (Hb02; FIGS. 3C, 3D, 3G and 3H.). Despite the reduction in blood volume (cHb), total tissue oxygenation (TOI) increased more during treatment with oxygen-infused water (FIGS. 3B and 3F). NIRS was used to measure the oxygen consumption rate of tissues in the foot. The tissue mass is heterogeneous; however, skeletal muscle is the predominate tissue in the NIRS probe location. The oxygen consumption rate was measured before and after treatment. In tap water treated feet it was 0.049±0.007 vs 0.056±0.008 ml.100 g⁻¹ min⁻¹; n=7 and for oxygen-infused water treated feet it was 0.052±0.003 vs. 0.050±0.002 ml.100 g⁻¹ min⁻¹; n=7. These values are not different and as a result show that treatment with oxygen-infused water does not alter the oxygen consumption rate of the tissue. The reduced blood volume and Hb and increased TOI do not result from a reduction in the foot's need for oxygen. The tissues still require the same amount of oxygen however, since some oxygen is absorbed across the skin, the tissue requires less blood flow to meet the oxygen requirement of the tissue.

### Calculated Oxygen Absorption Rate:

The average oxygen absorption rate when the foot was placed in 1 L of oxygen-infused water (dissolved O2 (DO); 36.5 mg·L¹) for 30 minutes was 1.2±0.1 μmole·cm²·min⁻¹ (Table 1). The DOI values typically obtained from the system range between 50 and 65 mg·L⁻¹. The starting values in this experiment were lower that this because some DOI was lost due to pouring the water from a graduated cylinder into the polyethelyne boot cover (FIG. 1B). For comparison, Fitzgerald (Physiol Rev. 1957; 37: 325-336) reviewed several earlier studies to 15 arrive at an uptake rate of 0.5 μmole·cm²·min⁻¹ for gaseous oxygen absorption by skin. Double the rate of oxygen absorption was measured using oxygen-infused water than previously measured for 100% oxygen air in contact with the skin. Oxygen-infused water is more effective for delivering oxygen across the skin than air.

TABLE 1
Measurement of foot oxygen absorption
from oxygen-infused water.

| | | Dissolved oxygen | | | Uptake |
|---|---|---|---|---|---|
| | | content (mg · L⁻¹) | | | (μmole · |
| Subject | FSA(cm) | t = 0 | t = 30 min | difference | com2 · hr⁻¹) |
| 1 | 606.7 | 46 | 31 | 15 | 1.5 |
| 2* | 630.2 | 32 | 23 | 8 | 0.8 |
| 3 | 630.2 | 32 | 24 | 8 | 0.8 |
| 4 | 629.4 | 31 | 19 | 12 | 1.2 |
| 5 | 553.2 | 32 | 17 | 15 | 1.7 |
| 6 | 585.8 | 42 | 36 | 7 | 0.7 |
| 7* | 772.9 | 32 | 23 | 9 | 0.7 |
| 8 | 772.9 | 46 | 23 | 23 | 1.9 |
| 9* | 694.1 | 32 | 19 | 13 | 1.2 |
| 10 | 694.1 | 39 | 23 | 16 | 1.4 |
| Mean | 619.2 | 36.5 | 23.9 | 12.6 | 1.2 |
| SEM | 27.4 | 2.0 | 1.8 | 1.6 | 0.1 |

Subjects with an asterisk(*) repeated the experiment on different days.
FSA is the calculated foot surface area.

Subjects were set up as in FIGS. 1A and 1C. Blood pressure cuffs were positioned around the ankle proximal to the medial and lateral malleous. Once properly positioned, the NIRS signal was given time to stabilize. Five minutes of resting baseline data was collected prior to inflating the blood pressure cuffs around each ankle to supra systolic pressures. The blood pressure cuffs acted as a tourniquet and stopped blood flow to the foot. Pilot work showed that 10 to 12 minutes of arrested blood flow was necessary to appreciably desaturate the haemoglobin trapped in the foot tissue of oxygen. After 12 minutes of occlusion, each subject was treated by continuing to soak on foot in tap water (control) and 5 soaking the other foot in oxygen-infused water. The experimental treatment lasted for 5 minutes and then the blood pressure cuffs were deflated to restore blood flow to the foot.

### Results

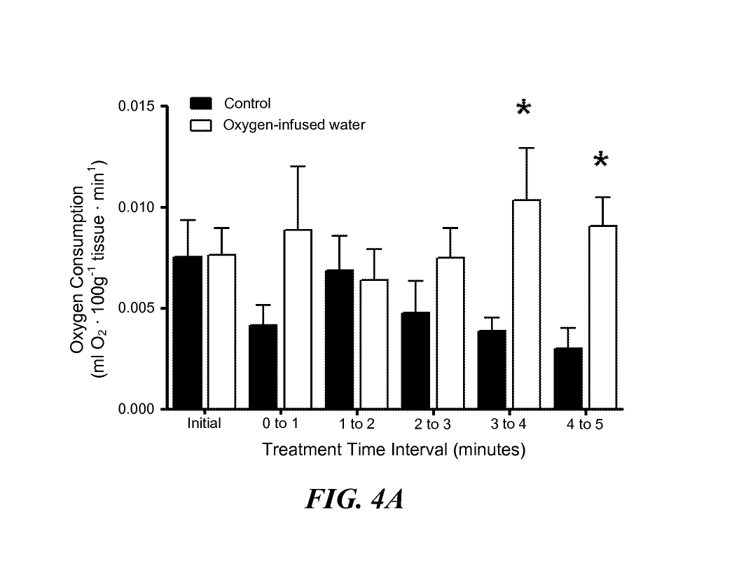

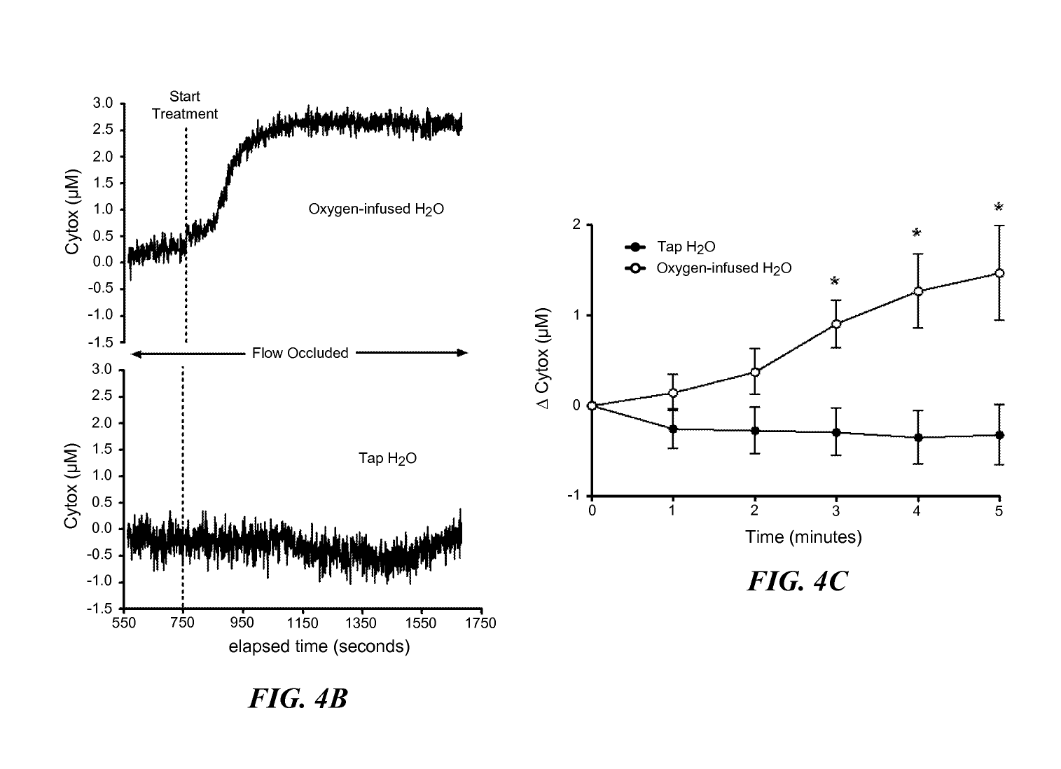

The oxygen consumption rate was measured for foot tissue about to be treated with either tap water or oxygen-infused water. The oxygen consumption rate measured before occlusion in tap water was 0.049±0.007 and 0.052±0.003 ml.100 g−1.min−1 (n=10) for the two experimental groups. At the end of the 12 minutes of blood flow occlusion the oxygen consumption rate fell to 0.0075±0.0018 and 0.0076±0.0013 ml.100 g−1.min−1 for both experimental groups. Exchanging the tap water for oxygen-infused water while maintaining flow occlusion resulted in the maintenance of a significantly higher oxygen consumption rate after 5 minutes of treatment (0.0078±0.0019 vs. 0.0034±0.0013 ml.100 g−1.min−1 FIG. 4A) when compared to feet that remained in tap water. Similarly, FIGS. 4B and 4C show an increase in oxidation of cytochrome oxidase aa3 in feet treated with oxygen-infused water during blood flow occlusion to the foot.

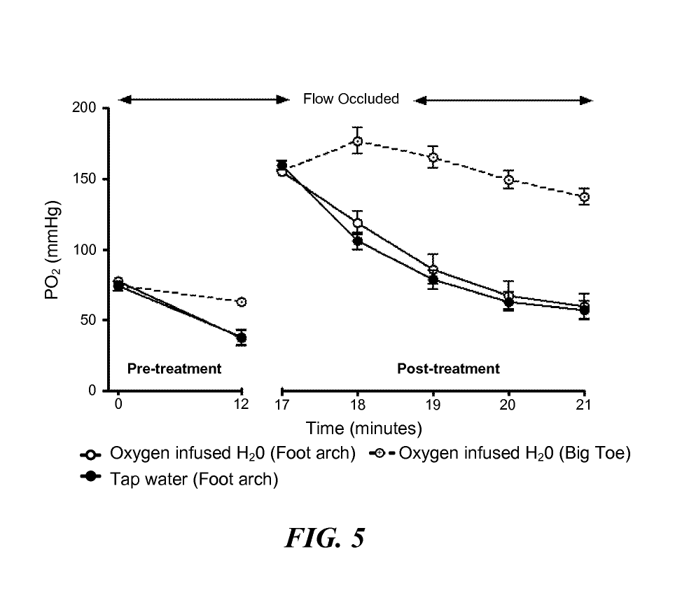

TcPO2 values recorded from the arch area of the foot declined in a similar pattern and magnitude while flow was occluded post treatment with tap water or oxygen-infused water (FIG. 5). Conversely, tcP02 values recorded on the plantar surface of the big toe were significantly elevated in oxygen-infused water treated feet when compared to values recorded on the foot arch (FIG. 5).

The toe lacks tissue with a high metabolic rate, thus the oxygen absorbed 5 during treatment is not immediately used to support metabolism. The toe tissue acts like a “sink”. In the arch area, the skin is fairly thin and lies directly over two large muscles in the foot that require significant amounts of oxygen even at rest. As a result, when blood flow is stopped and oxygen becomes limited in supply, any oxygen absorbed during treatment was immediately used to support metabolism in the muscle of the foot. In the toe, there was no immediate consumer of the absorbed oxygen and so the skin PO2 was able to increase (FIG. 5). It is also interesting to note that as little as 5 minutes of treatment significantly increased the PO2 in the toe's skin.

### Regional Differences in Oxygen Absorption on the Foot's Plantar Surface

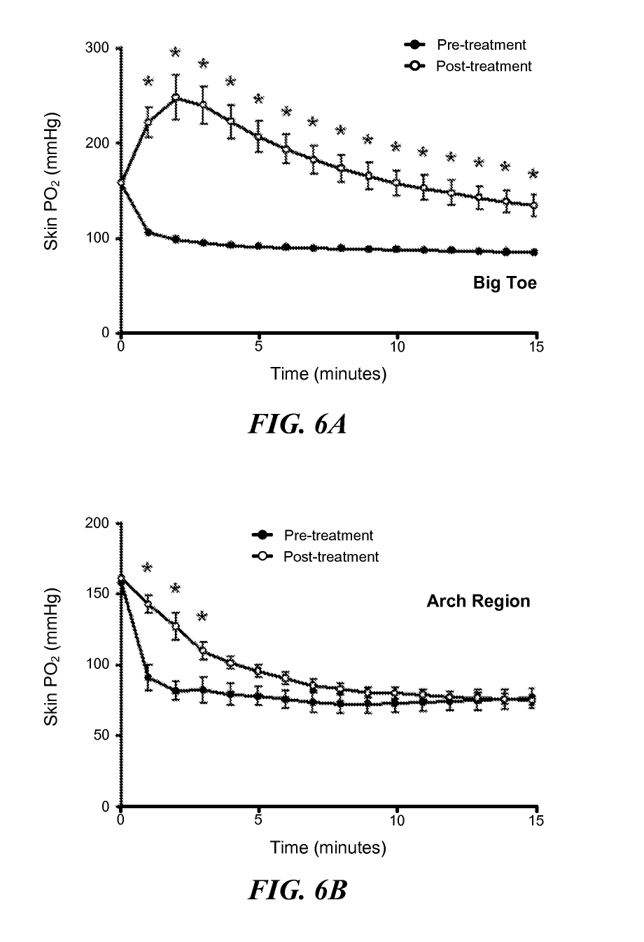

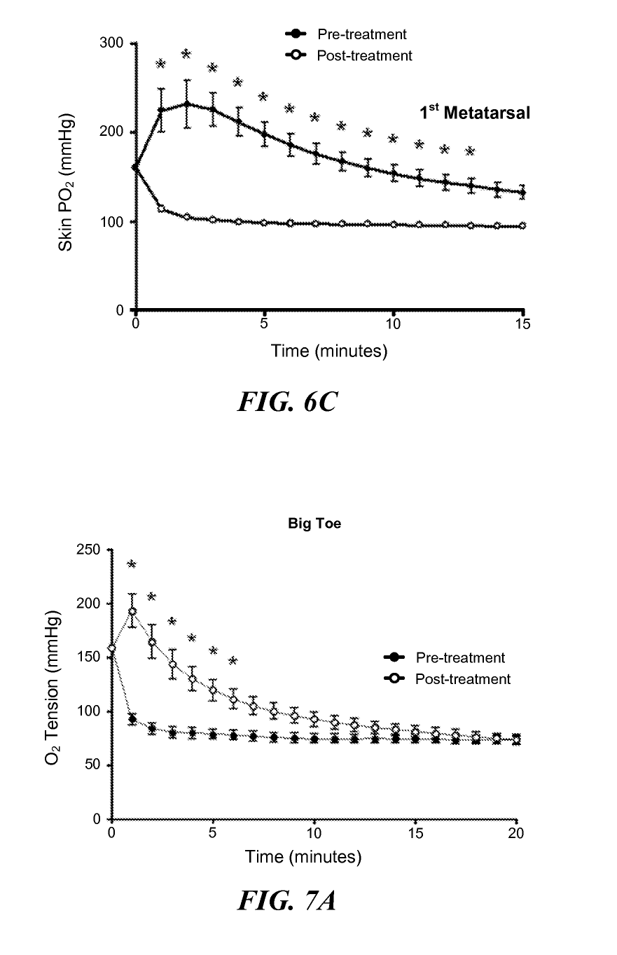

FIGS. 6A-C show tcPO2 data recorded from sensors placed on different regions of the foot; the plantar surface of the big toe (FIG. 6A); the plantar surface of the foot arch (FIG. 6B); and the plantar surface of the first metatarsus (FIG. 6C). These regions were selected primarily because the epidermal thickness and underlying tissue are quite different. The toe and metatarsal areas have thick epidermis and covers tissue with a lower metabolic rate while the arch area has a thin epidermis and metabolically active muscle underneath. Also, the plantar surfaces of the toe and metatarsus area are prone to diabetic foot ulcers and ulcers in these regions are most difficult to heal.

The tcPO2 data recorded pre and post treatment with oxygen-infused water show that areas with a thick epidermis and low metabolic rates retain or “soak up and store” the most oxygen during treatment. In these regions the PO2 in the skin remains significantly elevated 15 to 20 minutes post treatment. Even the arch region (FIG. 6B) had a significantly higher PO2 post treatment but it did not persist beyond 5 minutes. This is likely due to the thinner epidermis and because any oxygen that was absorbed was likely used to support oxygen consumption in the underlying muscle.

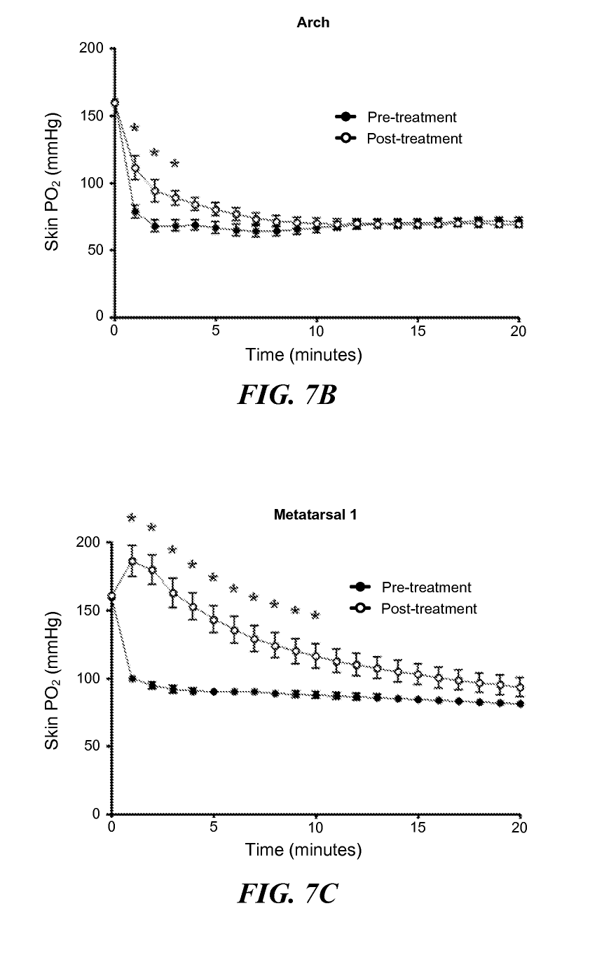

The same experiment was repeated in older adults (50 to 75 yrs). Although the findings are similar, the magnitude and persistence of the elevated tcPO2 measured after treatment was lower and shorter respectively (FIGS. 7A-C) in the big toe and metatarsus. It appears that aging slightly alters the amount of oxygen that skin can absorb from the oxygen-infused water. As a result, treatment dose (duration, frequency and number of treatments) may require adjustment if the patient with a diabetic foot ulcer is older.

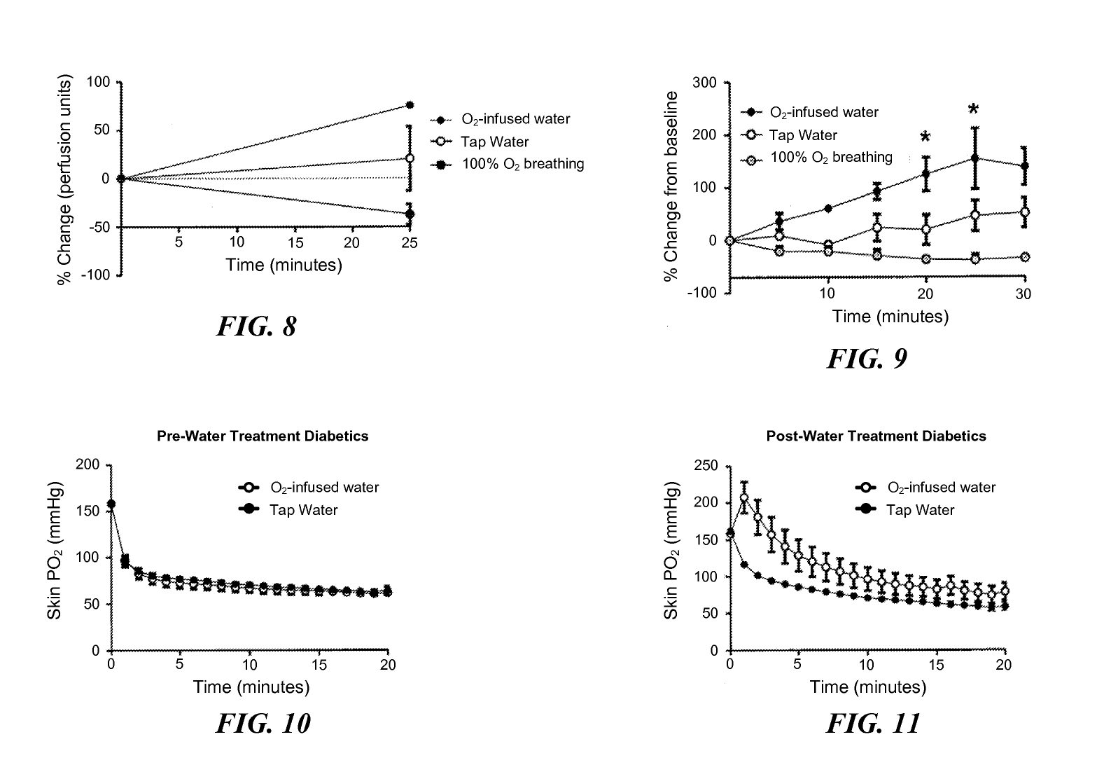

FIGS. 8-11 provide the results of additional experiments to study the effect of oxygen-infused water on the foot.

FIGS. 8 and 9 illustrate changes in blood perfusion resulting from oxygen-infused water, tap water and breathing 100% water. Specifically, FIG. 8 shows the percentage change from baseline of blood perfusion to the foot under three different conditions. The black, white and grey dots represent feet submerged in oxygen-infused water, tap water, and tap water while breathing 100% O2, respectively. No test of variance was conducted due to insufficient number of subjects, n=2.

When subjects submerge their feet in oxygen-infused water in accordance with the invention there is a 75% increase in blood perfusion. This is greater than the 20% increase observed in the paired foot submerged in tap water. When subjects submerge their feet in tap water and breathe 100% O2 there is a 50% reduction in blood perfusion. These findings are consistent with existing scientific literature, wherein excess O2 is considered to be a vasoconstrictor.

FIG. 9 also shows the percentage change from baseline of blood perfusion to the foot under three different conditions. The black, white and grey circles represent feet submerged in oxygen-infused water in accordance with the present invention, tap water, and tap water while breathing 100% O2, respectively. An asterisk identifies time points where blood perfusion is significantly (p<0.05) greater in the oxygen-infused water treated foot than the tap water treated foot. Significance was determined using a repeated measures ANOVA and Bonferroni post hoc comparison.

With an increased number of subjects (n=4), data continue to demonstrate an increase in blood flow in the oxygen-infused water treated foot. Toes soaked in tap water show a smaller increase in blood flow and breathing 100% O2 gas causes reduction in blood flow.

FIGS. 9 and 10 are the results from a diabetic group (n=6), showing oxygen transfer across the skin of the feet of diabetics. The O2 pressure in the big toe of each foot was measured for 20 minutes using tcPO2 sensors. Subjects then submerged one foot in tap water (pO2≈50 mmHg) and the other foot in O2-infused water (pO2≈1010 mmHg) for a 30 minutes treatment phase. Following the treatment phase, the O2 pressure in the big toe of each foot was again measured for 20 minutes.

FIG. 10 shows the O2 pressure in the big toes prior to treatment. The white and black circles represent O2-infused water and tap water respectively.

FIG. 11 shows the O2 pressure in the big toe following a 30 minute foot soak. The white and black circles represent O2-infused water and tap water respectively. Toes submerged in O2-infused water had greater O2 pressures than toes submerged in tap water. Soaking feet for 30 minutes in the oxygen-infused water increases the amount of O2 present in the tissue of individuals with diabetes. These findings are consistent with results from experiments conducted using healthy subjects aged 20-30 yrs and 50 yrs+. This demonstrates that diabetics are able to absorb O2 through their skin. O2 is a crucial element required in aerobic energy production and necessary for maintenance of cellular life. Individuals with diabetic foot ulcers generally suffer from compromised circulation. This causes a reduction in the nutritive blood flow, reducing O2 delivery to the wounded area. The ability to transport O2 to wounded tissue independently of the vascular system may benefit the healing process and prognosis of diabetic foot ulcers.

### Tonicity of the Treatment Water

One aspect to consider while developing a treatment protocol for diabetic foot ulcers is that open wounds can become macerated (waterlogged) when exposed to water for prolonged periods. The concern becomes damage to fragile newly developing tissue in the wound bed. At present, the treatment period is not long enough to cause waterlogged wound tissue. Also, wound exudates that accumulated during healing can cause injury to new tissue. Washing the wound to remove these products promotes healing. A topic in wound healing literature that remains equivocal is the tonicity of the solution used to irrigate and clean chronic wounds. Some wound experts advocate for sterile water while others advocate for isotonic salt solutions. The effect of adding NaCl to oxygen-infused water to create an isotonic 0.9% NaCl solution on water DOI values and the tcPO2 values obtained in skin soaked for 30 minutes in an oxygen-infused 0.9% NaCl solution was tested. No difference in DOI or tcPO2 values was observed in 5 experiments. Thus, using an isotonic oxygen-infused water solution is possible should it be necessary.

Particular embodiments of the subject matter have been described. Other embodiments are within the scope of the following claims.

1. A method of administering oxygen to a body site comprising:
providing an aqueous solution having a dissolved oxygen level of 18 mg/L or greater;
containing the aqueous solution in a receptacle suitable for at least partially submerging a portion of a body part;
applying the aqueous solution to the body part for a period of time allowing for oxygen transfer between the aqueous solution and at least some of the tissue over the body part;
removing the aqueous solution from contact with the body part, wherein the transcutaneous partial pressure of oxygen within a portion of the body part is elevated.

2. The method of claim 1 wherein the aqueous solution has a dissolved oxygen content of at least 35 mg/L.

3. The method of claim 1 wherein the oxygen is absorbed into the body part at a rate of 0.7 μmole·cm²·min⁻¹ or greater.

4. The method of claim 1 wherein the aqueous solution has an oxygen content of at least 45 mg/L and contact is maintained between the aqueous solution and the body part for at least 20 minutes.

5. The method of claim 1 wherein the aqueous solution is an oxygen infused NaCl solution.

6. A apparatus for wound treatment comprising:
a vessel shaped to accommodate a limb or extremity of a mammal, the vessel having sufficient volume to hold at least 0.5 liters of an aqueous solution in addition to the limb or extremity; and
an aqueous solution having a dissolved oxygen level of 18 mg/L or greater.

7. The apparatus of claim 6 further comprising an intake and an outtake for circulating the aqueous solution about the vessel.

8. The apparatus of claim 6 wherein the vessel is a water tight boot sized to hold 1.0 or more liters of an aqueous solution having a dissolved oxygen level of 30 mg/L or more.

9. The apparatus of claim 6 wherein the vessel may be closed in a manner to prevent leakage or spillage of the aqueous solution.

10. The apparatus of claim 6 wherein the aqueous solution is an oxygen infused NaCl solution.
